(19) United States
(12) Patent Application Publication
(10) Pub. No.: US 2017/0296638 A1
(43) Pub. Date: Oct. 19, 2017

(54) SYNERGISTIC COMPOSITIONS OF IMMUNOSTIMULATING RECONSTITUTED INFLUENZA VIROSOMES WITH IMMUNOPOTENTIATORS AND VACCINES CONTAINING THEM

(71) Applicant: CADILA HEALTHCARE LIMITED, Ahmedabad, Gujarat (IN)

(72) Inventors: Gaurav GUPTA, Ahmedabad - Gujarat (IN); Epifanio FICHERA, Ahmedabad - Gujarat (IN); Reinhard GLUECK, Ahmedabad, Gujarat (IN)

(73) Assignee: CADILA HEALTHCARE LIMITED, Ahmedabad, Gujarat (IN)

(21) Appl. No.: 15/508,223

(22) PCT Filed: Sep. 1, 2015
(86) PCT No.: PCT/IN2015/000340

§ 371 Date: Mar. 2, 2017

(30) Foreign Application Priority Data
Sep. 2, 2014 (IN) 2794/MUM/2014

(51) Int. Cl. A61K 39/008; C12N 7/00; A61K 39/39; A61K 39/00

(52) U.S. Cl. CPC A61K 39/008; C12N 7/00; A61K 39/39; A61K 2039/55572; C12N 2760/16034; A61K 2039/55588

(57) ABSTRACT

The present invention is directed to a preparation of an adjuvant system to achieve required level of humoral and cellular immune response against antigen of interest. The current invention provides an adjuvant system comprising an immunostimulating reconstituted influenza virosomes (IRIVs) and immunopotentiators. The current invention illustrates that an antigen is adsorbed or incorporated into IRIVs and further formulated with lipophilic adjuvant such as MPL or glucopyranosyl lipid adjuvant (synthetic analogue of MPL).

### FIELD OF THE INVENTION

The present invention is directed to a preparation of an adjuvant system to achieve required level of humoral and cellular immune response against antigen of interest. The current invention provides an adjuvant system comprising immunostimulating reconstituted influenza virosomes (IRIVs) and immunopotentiators. The current invention illustrates that an antigen can be adsorbed or incorporated into IRIVs and further formulated with an immunopotentiator, preferably a lipophilic adjuvant such as Mono Phosphoryl Lipid (MPL) or a glucopyranosyl lipid adjuvant (synthetic analogue of MPL, GLA).

### BACKGROUND OF THE INVENTION

A vaccine is prevention against any bacteria or viruses. It can act like an agent to protect your body from becoming sick. Basically, the difference between a vaccine and medication is that vaccine is prevention. The second is a treatment (the medication) that has to take periodically. Vaccinations are critical to building a child's immune system. babies are born with some immunity that they receive from their mothers, but that immunity begins to wear off after just a few months. Since they have not been exposed to disease, they have not had the opportunity to sufficiently build up their own immune system against vaccine-preventable diseases. Therefore, vaccine is necessary to develop for making healthy world by preventing each individual from vaccine-preventable diseases. One of the difficulties for the scientists in the development of therapeutic or prophylactic vaccines against infectious agents is to achieve the required protective level of immune response. The pure recombinant and synthetic antigens used in modern day vaccines are generally less immunogenic than older style live/attenuated and killed whole organism vaccines. The recombinant and synthetic antigens are preferred due to their simpler production and quality control, no other viral or external proteins, therefore less toxic, safer in cases where viruses are oncogenic or establish a persistent infection and feasible even if virus cannot be cultivated. One way to improve the quality of vaccine production is by incorporating immune-modulators or adjuvants with modified delivery vehicles viz. liposomes, immune stimulating complexes (ISCOMs), micro/nanospheres apart from alum, which is being used as gold standard. Adjuvants are used to augment the effect of a vaccine by stimulating the immune system to respond to the vaccine, more vigorously, and thus providing increased immunity to a particular disease. Adjuvants can be used for multiple purposes: to enhance immunogenicity provide antigen-dose sparing, to accelerate the immune response, reduce the need for booster immunizations, increase the duration of protection, or improve efficacy in poor responder populations including neonates, immune-compromised individuals and the elderly. Adjuvants are functionally defined as components added to vaccine formulations that enhance the immunogenicity of antigens in vivo. Adjuvants can be divided into two classes (delivery systems and immunopotentiators) based on their dominant mechanisms of action. Immunopotentiators activate innate immunity directly (e.g. cytokines) or through pattern recognition receptors (PRRs) (such as bacterial components), whereas delivery systems (e.g. microparticles and nanoparticles) concentrate the antigen and display antigens in repetitive patterns, target vaccine antigens to APCs (Antigen Presenting Cells) and help co-localize antigens and immunopotentiators. Thus, both immune-potentiators and delivery systems can serve to augment antigen-specific immune response in vivo.

Currently used adjuvants were developed using empirical methods, thus these are not optimal for many of the challenges in vaccination today. In particular, the historical emphasis on humoral immune responses has led to the development of adjuvants with the ability to enhance antibody responses. As a consequence, most commonly used adjuvants are effective at elevating serum antibody titers, but do not elicit significant Th1 (Type 1 T helper cells) responses or CTLs (Cytotoxic T-Lymphocytes). The ability of an adjuvant to qualitatively affect the outcome of the immune response is an important consideration, because the need for vaccines against chronic infections [e.g., Leishmania, HIV, hepatitis C virus (HCV), tuberculosis, human papilloma virus (HPV), malaria and herpes simplex virus (HSV) etc.] and cancer has shifted the focus to generation of cellular immune responses and adjuvants specifically geared towards eliciting this effect.

Major adjuvant groups include Alum based adjuvants, mineral salt adjuvants such as salt of calcium, iron grid zirconium, Complete Freund's adjuvant (CFA), Adjuvants emulsions such as Incomplete Freund's adjuvant (IFA), montanide, MF S9 and Adjuvant 65, bacterially derived adjuvants, their suitable combinations and the likes.

The benefits from adjuvant incorporation into any vaccine formulation have to be balanced with the risk of adverse reactions. Adverse reactions to adjuvants can be classified as local or systemic. Important local reaction include pain, local inflammation, swelling, injection site necrosis, lympho-adenopathy, granuloma formation, ulcers and the generation of sterile abscesses. Systemic reactions include nausea, fever, adjuvant arthritis, uveitis, eosinophilia, allergy, anaphylaxis, organ specific toxicity, immunosuppression or autoimmune diseases and liberation of different cytokines. Unfortunately potent adjuvant action is often correlated with increased toxicity as exemplified by the case of CFA (Complete Freund's adjuvant) which although potent is toxic for human use. Thus, one of the major challenges in adjuvant research is to gain potency while minimizing toxicity. The difficulty of achieving this objective is reflected in the fact that alum despite being initially discovered over 80 years ago, remains the dominant human adjuvant in use today.

The adjuvant properties of IRIVs are well known in the art, for example from WO 92/19267, wherein an adjuvant effect of the IRIVs for an antigen coupled thereto is disclosed.

However, although the use of virosomes as adjuvants has a number of advantages, for example low toxicity and high immunogenicity, one of the problems in current vaccinology is the lack of required immunogenicity of low immunogenic antigens. For subunit vaccines, it is highly desirable that a suitable combination of delivery systems, immunopotentiators and isolated antigens will be required to elicit optimal immune responses. In many cases, the addition of additional adjuvants to the virosomal formulation destroy the immunological property of the virosomal formulations due to high polarity of such adjuvants e.g. alum adjuvants deform the virosomes and squalene based adjuvants like MF-59 solubilizes virosomal membrane. Therefore, it is difficult to develop suitable adjuvant system comprising of delivery system and immunopotentiators.

Therefore, there is a need to develop an efficient immunopotentiating adjuvant system which can be used in the development of immunogenic composition and provide the desired humoral and cellular immune response against the antigen of interest.

Here, as per the present application, the inventors have developed a novel combination of immunostimulating reconstituted influenza virosomes with lipophilic adjuvants, wherein the lipophilic adjuvant is preferably a glucopyranosyl lipid adjuvant (Hereinafter, it is referred to as GLA), without destroying the immunostimulating effect of each system; on the contrary this adjuvant system provides surprising super stimulating effect.

### OBJECTS OF THE INVENTION

In one of the objects, the present invention provides an adjuvant system comprising suitable delivery system and suitable immunopotentiators.

In one of the objects, the present invention provides an adjuvant system comprising virosome as a delivery system and a suitable adjuvant as an immunopotentiator. Virosome according to the present invention is an immunostimulating reconstituted influenza virosomes (IRIV). IRIV according to the present invention is as disclosed in WO 92/19267. It is made up of (a) a mixture of phospholipids; (b) essentially reconstituted functional virus envelopes; and (c) an influenza hemagglutinin protein (HA) or a derivative thereof which is biologically active and capable of inducing the fusion of said IRIV with cellular membranes and of inducing the lysis of said IRIV after endocytosis by antigen presenting cells, preferably macrophages or B cells along with antigen of interest.

In a further aspect, the current invention provides an immunogenic composition comprising an antigen of interest along with the adjuvant system as described herein.

In a furthermore aspect, the immunogenic composition according to the present invention comprises (a) a mixture of a mixture of phospholipids; (b) essentially reconstituted functional virus envelopes; (c) an influenza hemagglutinin protein (HA) or a derivative thereof which is biologically active and capable of inducing the fusion of said IRIV with cellular membranes and of inducing the lysis of said IRIV after endocytosis by antigen presenting cells, preferably macrophages or B cells; and (d) an adjuvant and (e) an antigen of interest.

In one of the aspects, an antigen of interest includes infectious agents selected from a bacterium, a virus, a parasite and a fungus.

In another aspect, the current invention provides a method of preparing an adjuvant system comprising a delivery system and immunopotentiators.

In a preferred aspect, the current invention provides an adjuvant system comprising virosomes and lipophilic adjuvant preferably GLA.

In another aspect, the current invention provides use of an adjuvant system comprising virosomes and an adjuvant for the development of vaccine against infectious agent or carcinogenic or pathogenic agents.

In one of the aspects, the present invention provides a pharmaceutical composition for inducing an immune response against an immunogenic molecule (an antigen of interest) comprising an immunogenic composition with pharmaceutically acceptable carrier or excipient.

In a preferred aspect, the present invention provider vaccines containing immunogenic composition of the present invention for various antigens. These vaccines can be administered in conventional routes and dosages.

### DESCRIPTION OF THE FIGURES

FIG. 1 depicts Leish F3 protein expression in the host cell at one hour interval after induction by IPTG.

FIG. 2 depicts purified Leish F3 protein after diafiltration and sterile filtration.

FIG. 3 depicts that intact mass of the Leish F3 protein is in the expected range i.e. around 73 KDa.

FIG. 4 depicts that the identity of the Leish F3 protein has been confirmed by peptide mass fingerprinting.

FIG. 5 depicts humoral response against KMP 11 Leishmania antigen.

FIG. 6 depicts humoral response against LJL 143 Leishmania antigen.

FIG. 7 depicts humoral response against NH-SMT (Leish F3) Leishmania antigen:
(Provide a list of abbreviations for all the terms used in the specification)

### DETAILED DESCRIPTION OF THE INVENTION

The present invention is directed to a preparation of an adjuvant system to achieve adequate level of humoral and cellular immune response against antigen of interest.

In one of the embodiments, the adjuvant system comprises delivery system and immunopotentiators.

Delivery system according to the current invention is virosomes, preferably immunostimulating reconstituted influenza virosomes (IRIVs). IRIV according to the present invention is as disclosed PCT International application WO 92/19267. Immunopotentiators according to the current invention are adjuvants which are conventionally used in the preparation of vaccine to induce protection level of immune response against an antigen of interest.

Such adjuvant include Alum based adjuvants, mineral salt adjuvants such as salt of calcium, iron and zirconium. Complete Freund's adjuvant (CFA) Adjuvants emulsions such as Incomplete Freund's adjuvant (IFA), montanide, MF 59 and Adjuvant 65, bacterially derived adjuvants, lipophilie adjuvants, their suitable combinations.

Virosomes either adsorb or incorporates an antigen of interest to induce humoral response or cellular response against an antigen of interest respectively.

In a preferred embodiment, the present invention provides an immunogenic composition comprising an adjuvant system along with the immunogenic molecule. Such an immunogenic composition induces protecting level of immune response against an antigen.

In a more preferred embodiment, the current invention provides an immunogenic composition comprising (a) a mixture of phospholipids; (b) essentially reconstituted functional virus envelopes; (c) an influenza hemagglutinin protein (HA) or a derivative thereof which is biologically active and capable of inducing the fusion of said IRIV with cellular membranes and of inducing the lysis of said IRIV after endocytosis by antigen presenting cells, preferably macrophages or B cells; and (d) an adjuvant, preferably lipophilic adjuvant and (e) an antigen of interest.

The “mixture of phospholipids” described herein contains natural or synthetic phospholipids or a mixture thereof. At least it contains two different compounds selected from the group of glycero-phospholipids, such as phosphatidylcholine or phosphatidylethanolamine, and cholesterol.

The term “essentially reconstituted functional virus envelopes” refers to reconstituted influenza virus envelopes which are essentially devoid of the components which naturally occur inside of (below) the influenza virus envelope's membrane part. In a preferred embodiment the essentially reconstituted functional virus envelopes exhibit the form of a unilamellar bilayer. An example of such a lacking component is the matrix protein of the natural influenza virus envelope.

The term “biologically active HA or derivative thereof” as components of the IRIVs of the present invention refers to HAs or derivatives which substantially display the full biological activity of natural HA and are thus capable of mediating the adsorption of the IRIVs of the present invention to their target cells via sialic acid-containing receptors. Furthermore, such HA components can be recognized by circulating anti-influenza antibodies. This biological activity is an essential feature of the IRIVs of the present invention.

The term “lipophilic adjuvant” refers to TLR7 (Toll-like receptors) conjugated phospholipid i.e. Telormedix (herein after referred as TMX), Mono Phosphoryl Lipid A (herein after referred as MPL), GLA or combination thereof.

In one of the embodiments, an antigen of interest includes Leishmania, HIV, hepatitis C virus (HCV), tuberculosis and herpes simplex virus (HSV), malaria causing parasites, Human papilloma virus, and others like. Antigen of interest can be of hydrophilic or lipophilic nature. In the current invention, the lipophilic antigen is mixed with the formulated virosome; while hydrophilic antigen must be covalently linked to the surface of the virosome through cross-linkers. Linkers are well known in the art. A skilled person is able to select linker available in the art according to desired antigen. The linker can be cleavable linker, non-cleavable linker, acid-labile linkers, photo-labile linkers, peptidase-labile linkers, etc.

In a further embodiment, the current invention provides a method for the preparation and purification of antigen of interest. Antigen of interest can be prepared by conventional methods or techniques which include sequentially cloning the gene of interest, expression of the gene of interest, purification and characterisation of the protein obtained from the gene of interest. The steps mentioned herein above involve tools and techniques known in the art. A person skilled in the art can select such known techniques as per the requirement to achieve desired expression and purity of the antigen of interest.

Here, in the current invention, Leishmania antigens preferably Leish F3 (NH-SMT), VID 94, VID 99, VID 105, VID111, KMP 11, LJL 143, Leish F1, Leish F2 can be prepared by the steps mentioned above using known tools and techniques.

The gene of interest can be isolated from the genomic DNA of the parasite using techniques available in the art such as DNA isolation, PCR technology, etc, or can be chemically synthesized. Cloning of gene of interest includes insertion of gene of interest into vector by using restriction enzyme at different cloning site. Vectors used in recombinant technology are known in the art. Here, in the present invention, vectors can be selected from pET-29a(+) (Novagen), Pichia based vectors such as pPicz α, pPIC6, pGAPZ, pAO815 or other like vectors, mammalian cell based vectors such, as pOptiVEC-TOPO, pc DNA™ 3.1, etc. Here, according to the present invention, vectors can be preferably selected from pET-29a(+) (Novagen), pET-28a(+), pPicz α as per the different antigens of Leishmania.

Cloning is followed by transformation or transfection for further production of protein from the inserted gene of interest by using host cell system. The vector having gene of interest transforms or transfects it into host cell in which protein will be produced from inserted gene of interest. Host cell can be selected either prokaryotic such as E.coli or eukaryotic such as Pichia pastoris or mammalian cell such as CHO cell. Here, according to the present invention, host cell can be preferably selected from E.coli and Pichia pastoris.

Subsequently, high cell density fermentations can be carried out at the required scale by using methods known in the art. Such methods include batch, fed-batch and perfusion method. Here, in the present invention, fed-batch method is the preferred method for the large scale production of Leishmania antigen. Purification of protein obtained from the gene of interest preferably Leishmania antigen is carried out by using column chromatography techniques or filtration techniques or suitable combinations thereof. Column chromatography techniques includes ion exchange column chromatography, hydrophobic interaction column chromatography, affinity column chromatography, size exclusion column chromatography, mixed mode column chromatography and combination thereof. Filtration techniques mainly include ultrafiltration and diafiltration using various buffers such as phosphate buffer, tris buffer, citrate buffers and others like. A person skilled in the art can select appropriate purification technique available in the art to achieve desired level of purity. Here, according to the present invention, ion-exchange column chromatography technique is used to purify protein of interest, preferably protein of the target Leishmania antigens.

Here, in the present invention, protein characterisation has been done for antigen of interest by using intact mass and peptide mass fingerprinting techniques.

In another embodiment, the current invention provides a method of preparing an adjuvant system comprising a delivery system and immunopotentiators.

In a preferred Embodiment, the current invention provides a method for preparation of immunogenic composition comprising:
(a) Formulation of virosome;
(b) Adsorption of antigen to prepare modified virosome having antigen;
(c) Addition of adjuvant to the modified virosome having adsorbed antigen

Formulation of virosome is well described in PCT International application WO 92/19267.

To obtain humoral immune response against an antigen of interest, first virosomes are formulated, followed by adsorption with the desired antigen to obtain modified virosome having antigen. In case of the lipophilic antigen, antigens are mixed with the formulated virosome; while in the ease of hydrophilic antigen, antigens must be covalently linked to the surface of the virosome through cross-linkers.

To obtain cellular immune response, the antigens are added to the suspension of the virosome constituents and co-formulated subsequently. Such addition of antigen results into modified virosome having desired antigen either adsorbed to virosome or incorporated into virosome. Thus, the modified virosome according to the current invention is the virosome having desired antigen either adsorbed to virosome or incorporated into virosome.

According to the present invention, adjuvants, preferably lipophilic adjuvants are added to the above virosome formulation.

In a preferred embodiment, the current invention provides an adjuvant system comprising virosome and lipophilic adjuvant.

In a more preferred embodiment, the current invention provides an adjuvant system comprising IRIV and GLA or its derivative.

In another embodiment, the current invention provides a method of preparation of adjuvant system comprising:
(a) Formulation of modified virosome with lipophilic antigen or hydrophilic antigen
(b) Addition of adjuvant into modified virosome having antigen.

Here, according to the present invention, the adjuvant in the adjuvant system is selected from Alum based adjuvants, mineral salt adjuvants such as salt of calcium, iron and zirconium, Complete Freund's adjuvant (CPA), Adjuvants emulsions such as Incomplete Freund's adjuvant (IFA), montanide, MF 59 and Adjuvant 65, bacterially derived adjuvants, lipophilic adjuvants.

In a preferred embodiment, the adjuvant in the method of preparation of the adjuvant system is lipophilic adjuvant selected from Telormedix (herein after referred as TMX), Mono Phosphoryl Lipid A (herein after referred as MPL), GLA or combination thereof. In a more preferred embodiment, the adjuvant in the method of preparation of the adjuvant system is GLA or its derivative.

In another embodiment, the current invention provides use of an adjuvant system comprising virosome and immunopotentiators for the development of vaccine against infectious agent or carcinogenic or pathogenic agents.

In a preferred embodiment, the present invention provides combination of an adjuvant system with Leishmania antigen to induce protection level of immune response.

In one of the embodiments, the present invention provides a pharmaceutical composition for inducing an immune response against an immunogenic molecule (an antigen of interest) comprising an immunogenic composition with pharmaceutically acceptable carrier or excipient.

In a preferred embodiment, the present invention provides vaccines containing immunogenic composition of the present invention for various antigens. The vaccine comprises an antigen of interest and immunogenic composition as disclosed in the current invention which can elicit an immune response against target antigen. These vaccines can be administered in conventional routes and dosages.

In one of the embodiments, the present invention provides a method of stimulating immune response of a patient in need thereof comprising administering a suitable dosage of immunogenic composition as disclosed in the current invention.

Analytical Techniques used in the Current Invention

SDS PAGE: This is a technique used for the separation of proteins as per their molecular weight. Here the sample containing a mixture of proteins is run in an electric field in poly acrylamide gel of a particular sieve size and the proteins move differently according to their size and are thus separated. The band pattern obtained is compared with a molecular weight ladder to determine the molecular weight of the antigen.

BCA assay: This is a biochemical test for the quantification of proteins. The total protein concentration is exhibited by a color change of the sample solution from green to purple in proportion to protein concentration, which can then be measured using colorimetric techniques.

ELISA: This is Enzyme linked Immunosorbent assay where the seroconversion in the animals is measured by the interaction between specific antibodies with the corresponding antigens. The results obtained are measured by the intensity of the color the reaction mixture develops after reacting with the substrate used in the reaction. The results are measured in ELISA units.

### EXAMPLES

The following non-limiting examples describe the adjuvant system and its formulation with one of the antigen of interest which can be prepared as per the present invention. It will be appreciated that other immunogenic compositions with different antigens can be prepared and such immunogenic compositions are within the scope of a person skilled in the art and are to be included within the scope of the present invention.

### Example 1

### Preparation of Leish F3 (NH-SMT) Antigen and its Purification

### Leish P3 Antigen Preparation

LEISH-F3 was formed by the tandem linkage of two Leishmania open reading frames encoding the proteins namely nonspecific nucleoside hydrolase (NH) and sterol 24-c-methyltransferase (SMT). This step is applicable for fusion proteins.

### Cloning Details

The open reading frame of gene N (Nonspecific Nucleoside Hydrolase alias NH gene; GenBank XP_001464969.1) was PCR amplified from Leishmania infantum genomic DNA (Kumar et al. (2010) Am. J. Trop. Med. Hyg. 82; 808-813). Similarly, the open reading frame of gene S (Sterol 24-c methytranferase alias SMT gene; GenBank XP_001469832.1) was PCR amplified from Leishmania infantum genomic DNA. The PCR products were used as templates for fusion using splice-by-overlap PCR. Final fusion product was cloned into pET-29a (4) vector (Novagen). In case of non-fusion protein, single PCR product from the genomic DNA or chemically synthesized gene will be further cloned into vector. Here, pET-28a (+) or pPicz α can also be used as a vector. For example, a skilled person can use pOptiVEC-TOPO or pPicz α for LJL 143 antigen and pBT-28a (+) for KMP11 Leishmania antigens.

The recombinant plasmid was transformed into E. coli strain NS/HMS174 (DE3) for expression. A skilled person can use Pichia pastoris CHO cell line or other known mammalian cell line for the expression of recombinant antigen of interest.

### Expression of the Protein of Interest

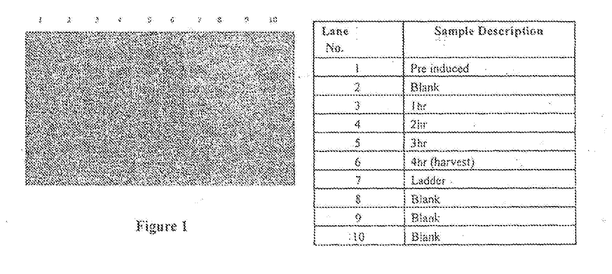

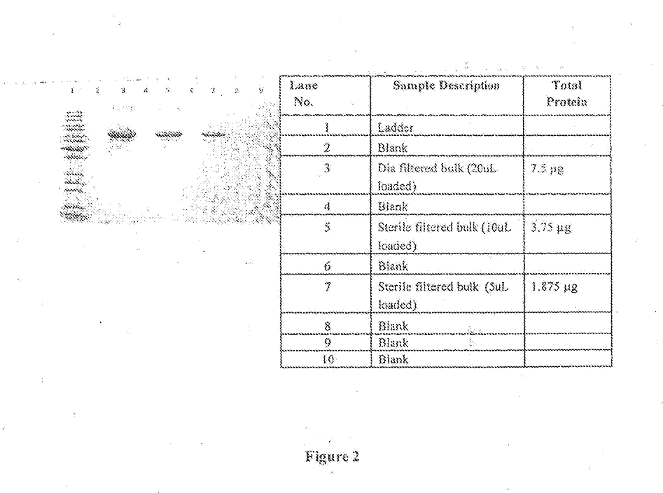

The clone was inoculated into the LB broth media to generate the seed for the further fermentation process. This seed was used to inoculate the fermenter containing defined media such as M9 for the growth of the host cell followed by the expression of the protein of interest. The hourly samples from the fermenter after induction by IPTG were lysed and loaded in a SDS PAGE gel and the expression of the protein of interest was confirmed as shown in FIG. 1. It shows that protein of interest is expressed at a desired level in the host system.

### Purification of Leishmania Protein

The cells harvested from the fermenter were lysed using a cell disrupter (French press) and the protein expressed in the form of inclusion bodies were isolated and purified using different buffer washes. The purified inclusion bodies were solubilized in chaotrophic agents like urea and guanidine hydrochloride. The solubilized protein containing solution was clarified and the supernatant was subjected to anion exchange chromatography. The elute obtained from this column were again subjected to anion exchange chromatography.

### Protein Refolding and Sterile Filtration

The purified protein was kept overnight in cold conditions in the presence of formulation buffer for the proper refolding of protein. The properly refolded protein was sterile filtered and stored.

The purified protein was analyzed for purity by SDS PAGE and the concentration of protein was determined by BCA (Bieinchronic acid assay). The gel image of the final protein is shown in FIG. 2. Single band at lanes 5 and 7 show that Leish F3 is purified up to a desired level using method of purification employed. The purified Leish F3 protein according to the present invention is more than 95% pure analysed by SDS PAGE. The yield of the purified protein is around 600 mg/L of fermentation broth.

### Protein Characterisation

The final purified protein was characterized by intact mass, peptide mass fingerprinting, and circular dichroism and fluorescence spectra.

### Intact Mass

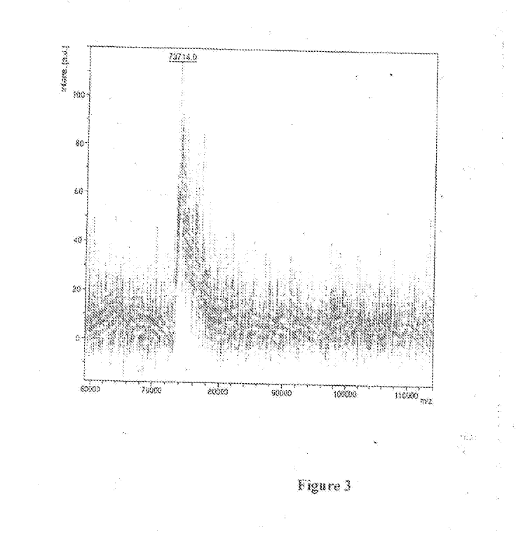

MALDI TOF analysis: was carried out to determine the actual molecular weight (mass) of the Leish F3 antigen. The drug substance of Leish F3 protein showed an intact molecular mass of 73714 Daltons. The data is shown in FIG. 3.

### Peptide Mass Fingerprinting

Peptide mass fingerprinting (PMF) is an analytical technique for protein identification in which the protein of interest is first cleaved into smaller peptides, whose absolute masses can be accurately measured with a mass spectrometer such as MALDI-TOF. The peptide masses are compared to a database containing known protein sequences using BLAST tool. The results are statistically analyzed to find the best match.

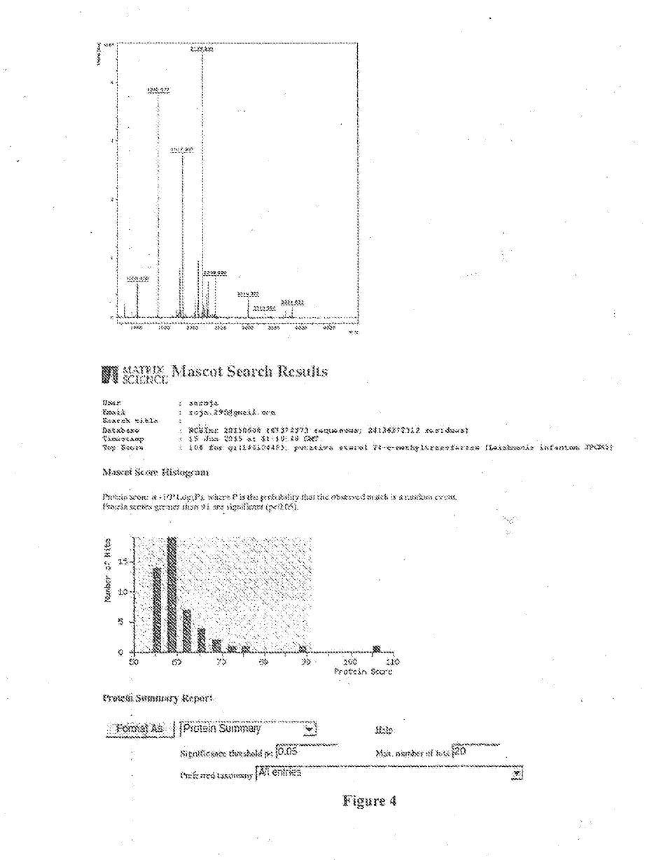

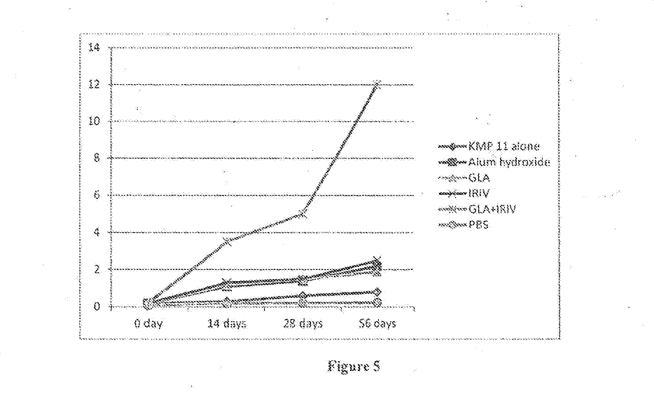

The PEPTIDE MASS FINGERPRINT of the Leish F3 sample gave a significant hit for ‘putative sterol 24-c-methyltransferasprotein’ from Leishmania infantum JPCM5 after the mascot search. It is shown as FIG. 4. The BLAST result obtained from MASCOT search shows that the Leish F3 protein of the current invention is significant according to the Mascot score of Histogram.

### Example 2

### Preparation of IRIV GLAs with Leishmania Virus Antigen Spontaneously Bound to their Surface

A pellet of purified influenza virus was solubilized using buffer and solvent system. The mixture was centrifuged and the supernatant containing the influenza spike proteins (HA) and viral phospholipids was added to the phospholipid mixture. The whole suspension was stirred for specific time at low temperature (4° C.). Subsequently, the suspension was applied to column which was equilibrated and elated with the same buffer as used for the preparation of the phospholipid dispersion. The sample volumes and column dimensions were such that a complete separation of IRIVs eluted at the void volume V O and cholate micelles was achieved. After the first chromatography, a second chromatography dialysis was performed. A purified antigen derived from Leishmania (NH-SMT or LJL-141 or KMP-11) containing was pelleted by ultracentrifugation. The IRIVs prepared above were added to the pellet. The Leishmania antigen spontaneously is adsorbed by Vander-Waals forces onto the surface of IRIVs.

The IRIVs—Leishmania complexes were carefully stirred for 24 hours at low temperature. Subsequently, a stable emulsion of GLA was added to the complex mentioned above. It was resulted into an immunogenic composition—IRIVs GLA adjuvanted with the Leishmania antigen. This immunogenic composition was analyzed to determine the humoral immune response by conventional technique.

### Example 3

### Preparation of IRIVs with Malaria Antigen Cross-Linked to the Membrane

The IRIVs were prepared according to Example 1 with the following alterations:

The malaria antigen molecules were attached to the IRIVs with a suitable cross-linker molecule.

Phosphoethanolamine (PE) was coupled with N-succinimidylpyridyl dithiopropionate (SPDP). The dried PE was redissolved is chloroform. Then triethylamine (TEA), followed by SPDP In dried methanol were added. The mixture was then stirred at room temperature under nitrogen for 1-2 hours until the reaction was complete (i.e. no more free PE). The reaction product was dried down on a rotary evaporator. The dried lipids were re-suspended in chloroform and were immediately applied on the top of a silicic acid chromatography column. The solution was poured into a 10 ml plastic syringe barrel plugged with glass fiber. The surplus was allowed to drain out and the syringe barrel was fitted with a plastic disposable three-way tap. After application of the lipids, the column was washed with chloroform. Finally, the column was elated with a series of chloroform-methanol mixtures. The pure derivative was then located by thin-layer chromatography (TLC) using silica gel plates developed with chloroform-methanol-water. The derivative runs faster than free PE and the spots are visualized by phosphomolybdate or iodine.

The fractions containing the desired product were pooled and concentrated by evaporation at reduced pressure in a rotary evaporator.

The malaria antigen (CSP antigen) was thiolated by the following procedure: purified malaria antigen was dissolved in phosphate buffer. Then, a SPDP solution at specified concentration in ethanol was mixed and was under stirring slowly added to the malaria protein solution with a Hamilton syringe to give a molar ratio of SPDP to protein of 15:1. The ethanol concentration was kept below 5% to prevent protein denaturation. The mixture was allowed to react for 30 minutes at room temperature (20° C.). After the reaction was stopped, the protein was separated from the reactants by gel chromatography, equilibrated with a solution containing sodium citrate sodium phosphate and 0.05 M sodium chloride.

The pretreated IRIVs and malaria antigens were coupled in the following manner: The IRIVs were prepared as described in Example 1. Instead of PE the PE-SPDP was used. Tire malaria—SPDP was reduced as follows: The pH of the malaria—SPDP—solution in citrate-phosphate buffer was adjusted to pH 5.5 by the addition of 1 M HCl 10 μl of a DTT solution, 2.5 M dithiothreitol (DTT, 380 mg/ml) in 0.2 M acetate buffer, pH 5.5 (165 mg of sodium acetate in 10 ml) was added for each ml of protein solution. The solution was allowed to stand for 30 min. Subsequently, the protein was separated from the DTT by chromatography on a column equilibrated with a PBS buffer, pH 7.0. In order to prevent oxidation of thiols all buffers were bubbled with nitrogen to remove oxygen. The protein fractions were also collected under nitrogen.

Finally, the IRIVs were mixed with the thiolated protein by stirring over night at room temperature.

Subsequently, a stable emulsion of GLA was added to the complex mentioned above. It was resulted into an immunogenic composition—IRIVs GLA adjuvanted with the malaria antigen.

### Example 4

### Incorporation of Leishmania Antigens into IRIVs GLA Complexes

The IRIVs were prepared according to Example 1 with the following alterations:
The Leishmania antigens were added to the suspension containing the influenza spike proteins (HA), viral phospholipids and the phospholipid mixture before column chromatography purification steps.

After last column chromatography step, the suspension was collected and added to an emulsified GLA suspension. It was resulted into an immunogenic composition—IRIVs GLA adjuvanted with the Leishmania antigen. This immunogenic composition was analyzed to determine the cellular immune response by conventional technique.

### Example 5

### Immunogenicity of IRIVs GLA Formulated KMP 11 Antigen of Leishmania

Groups of ten Balb/c mice had been immunized subcutaneously with the following formulations containing 2 μg of KMP 11 Leishmania antigen each:

| Group No. | Combination |
|---|---|
| 1 | KMP 11 alone (antigen control) |
| 2 | Alum hydroxide |
| 3 | GLA |
| 4 | IRIV |
| 5 | GLA-IRIV (adjuvant system |
| | according to present invention) |
| 6 | PBS (negative control) |

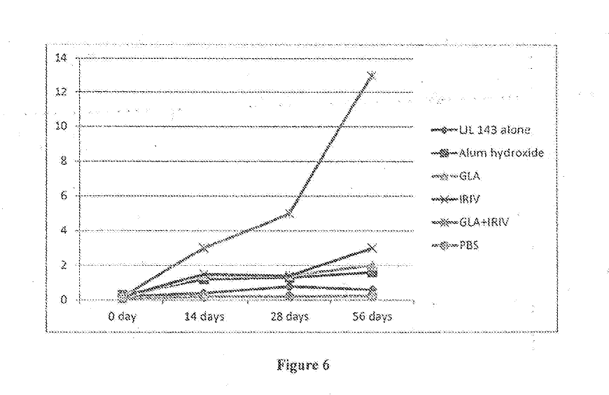

Humoral immune response has been monitored at various time intervals—0 day, 14 day, 28 and 56 day by ELISA determining IgG antibody. Results of this experiment are shown in FIG. 5. It shows that the composition comprising IRIVs GLA formulated KMP 11 according to the current invention showing synergistically higher immune response against KMP 11 Leishmania antigen as compared to other compositions of KMP11 antigen with various conventional adjuvants.

### Example 6

### Immunogenicity of IRIVs GLA formulated LJL 143 Antigen of Leishmania

Groups of ten Balb/c mice had been immunized subcutaneously with the following formulations containing 2 μg of LJL 143 Leishmania antigen each:

| Group No. | Combination |
|---|---|
| 1 | LJL 143 alone (antigen control) |
| 2 | Alum hydroxide |
| 3 | GLA |
| 4 | IRIV |
| 5 | GLA-IRIV (adjuvant system |
| | according to present invention) |
| 6 | PBS (negative control) |

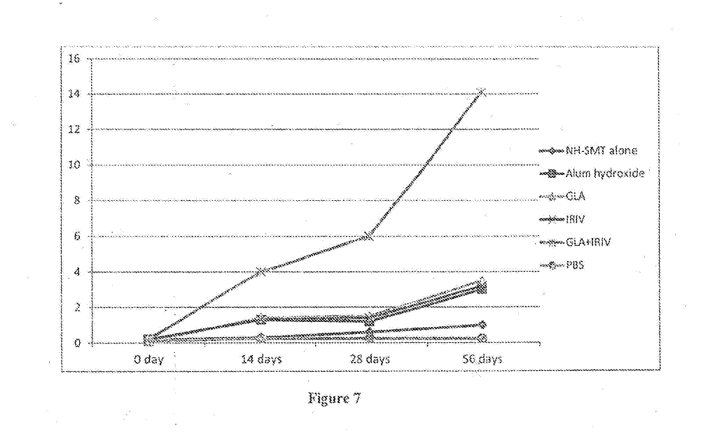

Humoral immune response has been monitored at various time intervals—0 day, 14 day, 28 and 56 day by ELISA determining IgG antibody. Results of this experiment are shown in FIG. 6. It shows that the composition comprising IRIVs GLA formulated LJL 143 according to the current invention is showing synergistically higher immune response against LJL 143 Leishmania antigen as compared to other compositions of LJL 143 antigen with various conventional adjuvants.

### Example 7

### Immunogenicity of IRIVs GLA Formulated NH-SMT (Leish F3) Antigen of Leishmania

Groups of ten Balb/c mice had been immunized spontaneously with the following formulations containing 2 μg of NH-SMT (Leish F3) Leishmania antigen each:

| Group No. | Combination |
|---|---|
| 1 | NH-SMT alone (antigen control) |
| 2 | Alum hydroxide |
| 3 | GLA |
| 4 | IRIV |
| 5 | GLA-IRIV (adjuvant system |
| | according to present invention) |
| 6 | PBS (negative control) |

Humoral immune response has been monitored at various time intervals—0 day, 14 day, 28 and 56 day by ELISA determining IgG antibody. Results of this experiment are shown, in FIG. 7. It shows that the composition comprising IRIVs GLA formulated NH-SMT (Leish F3) according to the current invention is showing synergistically higher immune response against NH-SMT (Leish: F3) Leishmania antigen as compared to other compositions of NH-SMT (Leish F3) antigen with various conventional adjuvants.

We claim:

1. An immunogenic composition comprising:
(a) an antigen; and
(b) an adjuvant system
wherein an adjuvant system comprises a delivery system and immunopotentiator(s).

2. The immunogenic composition as claimed in claim 1, wherein delivery system is virosome.

3. The immunogenic composition as claimed in claim 2, wherein virosome is an immunostimulating reconstituted influenza virosomes.

4. The immunogenic composition as claimed in claim 3, wherein immunostimulating reconstituted influenza virosomes comprising mixture of phospholipids, essentially reconstituted functional virus envelopes and biologically active HA or derivative thereof.

5. The immunogenic composition as claimed in claim 1, wherein immunopotentiator(s) is an adjuvant.

6. The immunogenic composition as claimed in claim 5, wherein the adjuvant is selected from alum based adjuvants, mineral salt adjuvants, Complete Freund's adjuvant (CFA), Incomplete Freund's adjuvant (IFA), montanide, MF 59 and Adjuvant 65, bacterially derived adjuvants, lipophilic adjuvants, hydrophillic adjuvants or their suitable combinations.

7. The immunogenic composition as claimed in claim 6, wherein mineral salt adjuvants is selected from salts of calcium, iron and zircon turn or their suitable combinations.

8. The immunogenic composition as claimed in claim 6, wherein lipophilic adjuvant is selected from Telormedix, Mono Phosphoryl Lipid A, glucopyranosyl lipid adjuvant and suitable combinations thereof.

9. A method for preparation of immunogenic composition comprising:
(a) Formulation of virosome;
(b) Adsorption or incorporation, of antigen to prepare modified virosome having antigen;
(c) Addition of adjuvant to the modified virosome having adsorbed antigen

10. The method as claimed in claim 9, wherein the virosome is an immunostimulating reconstituted influenza virosomes.

11. The method as claimed in claim 9, wherein adjuvant can be selected from hydrophilic adjuvants and lipophilic adjuvants.

12. The method as claimed in claim 11, wherein hydrophilic adjuvants is covalently linked to the surface of the virosome through cross-linkers.

13. An adjuvant system comprising:
(a) delivery system or vehicle; and
(b) immunopotentiator(s)

14. The adjuvant system as claimed in claim 13, wherein delivery system is virosome.

15. The adjuvant system as claimed in claim 13, wherein virosome is an immunostimulating reconstituted influenza virosomes.

16. The adjuvant system as claimed in claim 14, wherein immunopotentiator(s) is an adjuvant selected from Alum based adjuvants, mineral salt adjuvants, Complete Freund's adjuvant (CFA), Incomplete Freund's adjuvant (IFA), montanide, MP 59 and Adjuvant 65, bacterially derived adjuvants, lipophilic adjuvants, hydrophilic adjuvants.

17. The adjuvant system as claimed in claim 16, wherein lipophilic adjuvant is selected from Telormedix, Mono Phosphoryl Lipid A, glucopyranosyl lipid adjuvant and combination thereof.

18. An adjuvant system as claimed in claim 13 comprising:
(a) Virosome; and
(b) glucopyranosyl lipid adjuvant

19. The adjuvant system as claimed in claim 18, wherein virosome is an immunostimulating reconstituted influenza virosomes.

20. A method of preparation of adjuvant system as claimed in claim 13 comprising:
(a) formulating a modified virosome with lipophilic antigen or hydrophilic antigen
(b) Addition of adjuvant into modified virosome having antigen.

21. A pharmaceutical composition comprising immunogenic composition as claimed in claim 1, optionally with pharmaceutically acceptable carrier or excipient for inducing an immune response against antigen.

22. A vaccine containing immunogenic composition as claimed in claim 1, for inducing an immune response against antigen.

23. A method of stimulating immune response of a patient in need thereof comprising administering a suitable dosage of immunogenic composition according to any one of claims 1 to 22.

24. The immunogenic composition as claimed in any preceding claim, wherein antigen is selected from Leishmania, HIV, hepatitis C virus (HCV), tuberculosis and herpes simplex virus (HSV), malaria causing parasites, Human papilloma virus, preferably Leishmania antigen.

25. The immunogenic composition as claimed in claim 24, wherein Leishmania antigen is selected from Leish F3, VID 94, VID 99, VID 105, VID 111, KMP 11, LJL 143, Leish F1 and Leish F2; preferably Leish F3 or KMP 11 or LJL 143.
